(19) United States
(12) Patent Application Publication
(10) Pub. No.: US 2010/0215626 A1
(43) Pub. Date: Aug. 26, 2010

(54) MRSA Decolonization Kit (MDK)

(75) Inventor: Brian Patrick Murphy, Bloomington, IN (US)

Correspondence Address: Brian Murphy, 2027 S. Ramsey Dr., Bloomington, IN, 47401, US

(73) Assignee: Dr. Brian Patrick Murphy, Bloomington, IN (US)

(21) Appl. No.: 12/392,334

(22) Filed: Feb. 25, 2009

(51) Int. Cl. A61K 35/74

(52) U.S. Cl. 424/93.42

(57) ABSTRACT

The MRSA Decolonization Kit, or MDK, is a unique process available in a single kit to eliminate MRSA from individuals who have recently suffered an invasive infection from the potentially deadly bacteria and strengthen their body's ability to prevent recurrent infection. The kit provides patients with everything they need to remove MRSA from their skin flora and replace it with a far less dangerous bacteria, Staphylococcus Epidermidis. The kit has detailed instructions on using the bactericidal ointments and cleansers followed by the application of Sepiderm Lotion that contains viable, healthy Staphylococcus Epidermidis. It also includes detailed instructions on ridding the home environment of the dangerous bacteria as well as the vital component of personal hygiene to minimize repeat contamination with the bacteria.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAMLISTING COMPACT DISC APPENDIX

Not Applicable

### BACKGROUND INFORMATION

MRSA, Methicillin resistant staphylococcus aureus, is now the most frequent cause of skin and soft tissue infections presenting to the emergency room (1). In a recent study published in Emerging Infectious Diseases it was reported that in the time period between 1999 and 2005 the number of hospitalizations due to MRSA infection increased by 62% and the number of deaths related to the bacteria doubled (2).

According to the CDC website the prevalence of these strains in the community will likely continue to grow. This increasing prevalence inevitably will cause an upsurge of the use of the few antibiotics that remain effective against this dangerous pathogen, adding further to the problem of antibiotic resistance. The effective treatment of these infections will require stronger antibiotics for longer durations; as a result, the resistance of these organisms will continue to escalate.

The solution to the growing MRSA epidemic will not be found through the development of new antibiotics but instead through education, improved hygiene and decreasing the number of carriers of this harmful pathogen. The process outlined herein is a step towards reversing the trend of increasing infection rates and escalating morbidity and mortality associated with MRSA. The simple process decreases the number of harmful bacteria and strengthens individual's natural defenses against invading organisms.
1. Moran G J, Krishnadasan A, Gorwitz R J, et al. Methicillin-resistant S. aureus infections among patients in the emergency department. N. Engl. Journ. of Med. 2006; 355(7):666-674.
2. Klein E, Smith D L, Laxminarayan R. Hospitalizations and deaths caused by methicillin-resistant Staphylococcus Aureus, United States, 1999-2005. Emerging Infectious Diseases. December 2007;13(12):1840-6

### BRIEF SUMMARY OF THE INVENTION

The MRSA Decolonization Kit, MDK, is an original process of using a body treating composition to rid an individual of harmful, resistant bacteria and replace them with healthy skin flora susceptible to commonly used antibiotics. In medicine, once a patient has been diagnosed with a MRSA infection it is common practice to consider that individual a carrier from that point forward. The MDK can be used after a MRSA infection has been diagnosed and appropriately treated with antibiotics in an attempt to rid an individual of the harmful opportunistic pathogen. It is a two step process that is to be repeated over a period of 7 days. The first step is decolonization. The individual colonized with MRSA will first wash their entire body with a 4% chlorhexidine gluconate solution and apply a liberal amount of mupirocin ointment to both nares. The second step is recolonization. The individual then applies Sepiderm, a lotion designed to effectively deliver Staphylococcus Epidermidis to the skin. This two step process is then repeated over a 7 day time frame. Also, during this time frame the patient is given very detailed and specific instructions on sterilizing their home environment.

MRSA is becoming more resistant to antibiotics and causing harm to patient in an ever increasingly wide spectrum of demographics. The MDK can help curb this trend by strengthening the best defense the human body has against invading organisms, healthy skin. The MDK is designed to kill bacteria on the host and replace them with antibiotic susceptible Staphylococcus Epidermidis, a bacteria found in normal skin flora. If the host is colonized with this far less harmful bacteria they can better defend themselves against an invasion by a more harmful one.

### BRIEF DESCRIPTION OF THE SEVERAL VIEWS OF THE DRAWING

Not Applicable

### DETAILED DESCRIPTION OF THE INVENTION

The MDK will be sold as a kit available by prescription only. The kit will include the following:
A detailed instruction manual
One tube of mupirocin ointment and 7 cotton swab applicators
One bottle of 4% chlorhexidine gluconate wash
One bottle of Sepiderm Lotion
   The key to the MDK is the Sepiderm Lotion. It is made by mixing the following proportions based on percent weight.
1. 75% Staphylococcus Epidermidis nutrient broth that is commercially available with the appropriate permits
2. 16% Mineral Oil/Shea Butter
3. 5% Emulsifying wax also commercially available
4. 4% Stearic acid

The instruction manual will have three components, personal care guidelines, disinfecting the household and details on using the two step decolonization/recolonization materials.

The first is personal care guidelines.
Cover your nose and mouth when you sneeze or cough. Wash hands immediately afterwards
Keep your fingernails and toenails short to minimize safe harbor for bacteria
Change your sheets regularly
Do not reuse towels or wear clothes more than once without washing
Do not share any personal items i.e. toothbrush, razor, deodorant
Wash hands frequently and use an over the counter moisturizing lotion after Disinfecting the Household
Use a disinfectant purchased at the grocery store to frequently wash hard surfaces.
Focus on items that are frequently touched: door knobs, light switches, kitchen counters, cell phones, toilets, sinks, computer keyboards
Where disposable gloves and wipes when cleaning. After use throw away gloves and wipes and immediately wash hands with an antibacterial soap.
   Using the decolonization/recolonization materials

Wash daily with the cleansing solution making sure all areas of exposed skin are thoroughly scrubbed with a wash cloth. Avoid contact with mucous membranes, such as your eyes and mouth. Wash the wash cloth immediately after use.
After washing use the supplied cotton swabs to liberally apply the mupirocin ointment to the inside of each nostril making sure that all inner surfaces have been contacted. Dispose of the cotton swab immediately after use
Liberally apply the Sepiderm lotion to all exposed areas of the skin and massage until absorbed. Again, avoid contact with mucous membranes such as the eyes and mouth.
Repeat the process for 7 days. During this time period follow the above personal care guidelines, household disinfecting recommendations very closely.

1. The Sepiderm lotion is a unique mixture that effectively delivers viable bacteria to the skin allowing colinization with the Staphylococcus Epidermidis bacteria it contains.
